(19) United States
(12) Patent Application Publication
(10) Pub. No.: US 2016/0038352 A1
(43) Pub. Date: Feb. 11, 2016

(54) DISPOSABLE BAG DIAPER ATTACHMENT

(71) Applicant: Brigitte Greene, Baltimore, MD (US)

(72) Inventor: Brigitte Greene, Baltimore, MD (US)

(21) Appl. No.: 14/815,925

(22) Filed: Jul. 31, 2015

Related U.S. Application Data
(60) Provisional application No. 62/031,781, filed on Jul. 31, 2014.

(51) Int. Cl. A61F 13/551

(52) U.S. Cl. CPC A61F 13/5512; A61F 2013/55125

(57) ABSTRACT

A disposable diaper has a disposal bag contained in the diaper, wherein the diaper comprises an outer layer and a pocket in the outer layer, and wherein the pocket contains a disposal bag. The disposal bag can be unfolded to receive a used diaper for easy and sanitary disposal.

### CLAIM OF PRIORITY

This patent application claims priority under 35 USC 119 (e) (1) from U.S. Provisional Patent Application Ser. No. 62/031,781 filed Jul. 31, 2014, of common inventorship herewith entitled, “Disposable Bag that Attaches to Diapers,” which is incorporated herein by reference as though the same were set forth in its entirety.

### FIELD OF THE INVENTION

The present invention pertains to the field of diapers, and more specifically to the field of disposable diapers.

### BACKGROUND OF THE INVENTION

The prior art has put forth several designs for disposable diapers. Among these are:

US Patent 2006/0282056 to Michael L. McDonald describes an exemplary embodiment providing one or more improvements that include a fluid impermeable bag attached to or integral with a disposable diaper. The bag has closure elements on the outside of the front and back panels of the uninverted bag. After use, the bag is inverted over the soiled diaper and the closure elements interact, thereby closing and sealing the soiled diaper inside the bag. In some embodiments, the bag is covered by a removable fabric element such as a gauze panel with decorative patterns.

US Patent 2002/0065500 to Paul J. Rossi describes a tear open enclosure housing a disposable plastic bag that is located directly on the diaper pull up allowing the diaper to be stored in this disposable plastic bag and discarded at a later time. The Disposable Plastic Bag is attached directly to the diaper pull up with a flexible nontoxic adhesive or a fastening tab and then enclosed by an accessible perforated enclosure. The Disposable Plastic Bag is also attached to a nonperforated enclosure, which then is covered by an accessible perforated enclosure. This entire enclosure is attached to the diaper pull up.

US Patent 2002/0004656 to Farzana Khan and Jacquel Smith describes a disposable diaper system for infants, babies, senior citizens or physically challenged individuals. The system includes a diaper formed as a substantially rectangular blank having tabs at the corners and elastic leg engaging areas to prevent or minimize leakage and to provide a secure and comfortable fit. For hygiene purposes, the system includes a first and second storage compartment coextensive therewith for storing an integrated disposable bag and gloves for reducing feces or body fluid contamination during diaper removal. The system is configured with materials which provide a reduced overall package volume including both the bag and gloves.

U.S. Pat. No. 7,569,038 to Bernard Louis Salem, Jr. describes a disposable diaper having at least one pocket or pouch for containing an attached waste bag. The waste bag has an upper end adapted for sealable closure. The disposable diaper includes an elongated slit which provides entry into an interior cavity that is adapted to contain an auxiliary waste bag. The disposable diaper is embellished with ornamentation, decorative patterns and symbols so as to represent a particular theme.

None of these prior art references describe the present invention.

### SUMMARY OF THE INVENTION

It is an object of the present invention to provide a line of baby and adult diapers that incorporates a plastic bag directly in the diaper itself.

### BRIEF DESCRIPTION OF THE DRAWINGS

FIG. 1 is a prototypical front view of the present invention.

FIG. 2 is an illustrative prototypical front view of the present invention showing the tape that secures the diaper on the person.

FIG. 3 is an illustrative prototypical back view of the present invention showing a compartment opening that houses a disposable bag.

FIG. 4 is an illustrative prototypical back view of the present invention showing the disposable bag in the housing compartment.

### DETAILED DESCRIPTION OF THE INVENTION

The anticipated arrival of a new baby is a very special time. While the expectant mother's belly swells, the joy of the coming birth becomes more meaningful with each passing day. Nothing truly prepares parents for hard work involved once a baby is born. Fevers and tempers brought on by colic and teething render parents concerned and frustrated, and sleepless nights rocking a restless infant leave them exhausted. Snuggling a baby close to one's heart, comforting them when they cry and seeing a toddler take their first few steps all serve to make the pitfalls of parenting more than worthwhile. Over time, parents adjust to schedules and needs of their newborns while sleepless nights and chaotic days give way to a more relaxed routine.

One daunting thing to which most new parents never fully adjust is an arsenal of equipment that is kept on hand to keep a baby both comfortable and happy. Fresh bottles of milk or sippy cups of juice and water are stored within easy reach to satisfy an infant's empty belly or quench a toddler's thirst. Jars of baby food line cabinet shelves and fruit snacks and baby cookies provide a healthy treat for a grumbling child. Hard teething biscuits and cold teething rings massage sore gums and calm a crying baby through the onset of first teeth, while small toys, rattles or a special blanket entertain and comfort a cranky child. A never ending supply of disposable diapers ensure that a baby's tender bottom is kept clean, dry and free of irritating and painful diaper rash. Disposable diapers offer a hygienic means of containing a child's normal bodily waste while preventing stains and safeguarding expensive clothing. Particularly challenging is disposing of the soiled diaper when one takes their baby away from home. After removing a diaper from the child, the adult typically rolls the diaper into a compacted form and then attempts to locate a convenient means of throwing it away while out and about.

The present invention, hereinafter referred to as the Disposable Bag Diaper Attachment or the Disposable Bag for Diapers, is an enhancement to baby and adult diapers that incorporates a plastic bag directly in the diaper itself. The disposable diaper has a disposal bag contained in the diaper, wherein the diaper comprises an outer layer and a pocket in the outer layer, and wherein the pocket contains a disposal bag. The disposal bag can be unfolded to receive a used diaper for easy and sanitary disposal.

Please refer to the Figures.

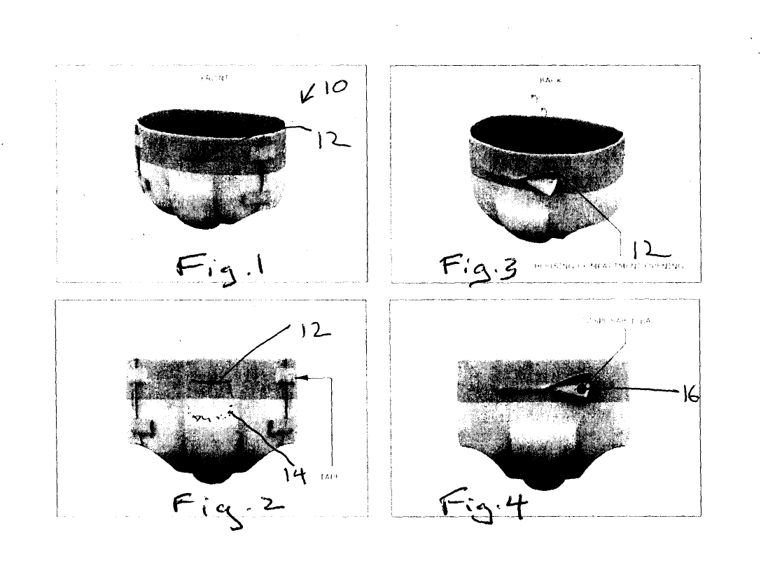

FIG. 1 shows a disposable diaper 10 having a slit opening 12.

FIG. 2 shows a disposable diaper 10 having a slit opening 12, and pocket 14 (in phantom).

FIG. 3 shows the diaper 10 having slit opening 12 and bag 16 protruding from the slit opening of pocket 14.

The Disposable Bag for Diapers provides parents and caregivers, as well as adults who wear such garments, with a simple and sanitary means of disposing of soiled diapers after use. Similar in configuration to traditional diapers, each diaper in the present invention's line is manufactured with absorbent cotton like material and backed with soft flexible plastic. Self affixing tape fasteners are positioned on either hip for securing the diaper about a child or adult's waist. The present invention is manufactured in a range of sizes for wear by newborns, infants, toddlers, older children and adults.

In an embodiment, the bag can be comprised of furan based material, or other compostable and bio-degradable material.

Unique to this product and what sets it apart from a common diaper is a titular bag. A rectangular pouch is integrally attached to the top rear waistband section of each diaper. Compacted inside the diaper in an accordion fashion, a plastic bag is readily available to pull from the pouch in the manner one removes a tissue from a box. Possessing an appropriate length, width and depth to accommodate the diaper to which it is attached, the bag is sized to fully encompass the soiled garment within its confines. In an embodiment, the bag is approximately twelve inches in length an six inches in width and has a pleated bottom to expand the effective opening of the bag to receive the diaper.

One needs only to remove the diaper, roll it in the usual fashion, and deftly remove the disposable bag. The diaper is inserted into the bag, cleanly ready for placement in a garbage receptacle.

The Disposable Bag for Diapers line is packaged in a durable, plastic bag containing approximately twenty diapers per package. The present invention also is individually packaged and available in vending machines found in public rest room facilities, providing busy parents with all the essentials necessary to care for their child when on the go. Compact in size, the Disposable Bag for Diapers is tossed easily in a purse or glove compartment, ensuring that busy parents are always prepared for unexpected accidents. Although designed with household consumers in mind, the present invention is highly functional and effective for baby sitters and child care providers, enabling these professionals to cleanly and easily care for their charges. The Disposable Bag for Diapers is extremely functional for older consumers who suffer from incontinence, a condition that necessitates repeated changing of soiled garments.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A disposable diaper having a disposal bag contained in the diaper, wherein the diaper comprises an outer layer and a pocket in the outer layer, wherein the pocket contains a disposal bag, and wherein the disposal bag is capable of being unfolded and contain a used diaper.

2. The disposable diaper of claim 1, wherein the disposal bag is comprised of furan based material, or other compostable and bio-degradable material.

3. The disposable diaper of claim 1, wherein the pocket is a rectangular pouch which is accessed by a slit opening in the outer layer of the diaper.

4. The disposable diaper of claim 3, wherein the slit opening is located in the top front or rear waistband section of the diaper.

5. The disposable diaper of claim 1, wherein the bag inside the pocket is folded in accordion folds.

6. The disposable diaper of claim 3, wherein the bag is sized to an appropriate length, width and depth to accommodate the diaper.

7. The disposable diaper of claim one wherein the bag is approximately twelve inches in length an six inches in width and has a pleated bottom to expand the effective opening of the bag to receive the diaper.
